(19) United States
(12) Patent Application Publication
(10) Pub. No.: US 2023/0250978 A1
(43) Pub. Date: Aug. 10, 2023

(54) MICRO AIR AND SURFACE SPRAYER

(71) Applicants: Douglas James Sprague, Airdrie (CA); Steven Seth Gareleck, Marietta, GA (US); Brian Christopher Domrese, Charleston, SC (US)

(72) Inventors: Douglas James Sprague, Airdrie (CA); Steven Seth Gareleck, Marietta, GA (US); Brian Christopher Domrese, Charleston, SC (US)

(21) Appl. No.: 17/650,083

(22) Filed: Feb. 6, 2022

(51) Int. Cl. F24F 6/14; B05B 15/40; B05B 17/06; A61L 2/18; A61L 9/14

(52) U.S. Cl. CPC F24F 6/14; B05B 15/40; B05B 17/0653; A61L 2/18; A61L 9/14; F24F 2006/143; F24F 2110/20

(57) ABSTRACT

A system having an enclosure for containing a solution. An atomizer is in communication with an interior of the enclosure and the solution for generating an aerosolized particle mist within the interior of the enclosure. A positive pressure device is in communication with the interior of the enclosure for generating a positive pressure within the enclosure relative to an atmosphere outside of the enclosure. An ejection nozzle having a tortuous path is provided, wherein an entrance of the tortuous path is in communication with the interior of the atomizer and an exit of the tortuous path is in communication with the atmosphere outside of the enclosure.

### BACKGROUND

The present invention generally relates to particle generation and more specifically, to a micro air and surface sprayer.

Coronavirus Disease 2019 (“COVID-19”) is spreading throughout the world caused by the spread of a novel coronavirus called SARS-CoV-2. With the rapid spread of the disease, greater importance has been placed on disinfection of air, surfaces, and subsurfaces. As coronavirus, and other pathogens, spread in the environment, removal of these pathogens has become a top priority. Various methods and devices have been used to attempt to destroy pathogens, but each of the currently available methods, such as spraying disinfectant or utilizing ultraviolet light, fails to reach the majority of pathogens that are not disposed on top surfaces in the environment. The presence of these less accessible pathogens creates a hazard for anyone present in the environment.

### SUMMARY

Embodiments of the present invention are directed to a system having an enclosure for containing a solution. An atomizer is in communication with an interior of the enclosure and the solution for generating an aerosolized particle mist within the interior of the enclosure. A positive pressure device is in communication with the interior of the enclosure for generating a positive pressure within the enclosure relative to an atmosphere outside of the enclosure. An ejection nozzle having a tortuous path is provided, wherein an entrance of the tortuous path is in communication with the interior of the atomizer and an exit of the tortuous path is in communication with the atmosphere outside of the enclosure.

Further embodiment of the present invention are directed to a method of providing a micro-aerosol to an atmosphere. The method includes generating an aerosolized particle mist within an enclosure and providing a positive pressure to the aerosolized particle mist within the enclosure. The method eliminates a substantial majority of particles larger than about 2.5 microns from the aerosolized particle mist using a tortuous path prior to releasing the aerosolized particle mist into the atmosphere as the micro-aerosol.

Further embodiment of the present invention are directed to a method of disinfecting an atmosphere and a surface of contamination. The method includes generating an aerosolized particle mist within an enclosure and providing a positive pressure to the aerosolized particle mist within the enclosure. The method eliminates a substantial majority of particles larger than about 2.5 microns from the aerosolized particle mist using a tortuous path prior to releasing the aerosolized particle mist into the atmosphere as the micro-aerosol.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts an orthogonal view of a micro air and surface sprayer (“MASS”) according to an embodiment of the present invention;

FIG. 2 depicts a cutaway view of the MASS according to an embodiment of the present invention;

FIG. 3 is a top view of the MASS according to an embodiment of the present invention;

FIG. 4 is a side view of the MASS according to an embodiment of the present invention;

FIG. 5 is a top down, cut-away view of the MASS, showing the atomizer with a plurality of ultrasonic nozzles;

FIG. 6 is an orthogonal, cut-away view of an ejection nozzle having a tortuous path according to an embodiment of the present invention;

FIG. 7 is an additional orthogonal, cut-away view of an ejection nozzle having four internal grates according to an embodiment of the present invention;

FIGS. 8 and 9 are cut-away view of ejection nozzles having four and eight internal grates, respectively, according to embodiments of the present invention;

FIG. 10 is an orthogonal view of the internal grate according to an embodiment of the present invention;

FIG. 11 is a piping and instrumentation diagram of a MASS according to an embodiment of the present invention; and

FIG. 12 is a flowchart of a method 1200 for generating a micro-aerosol according to an embodiment of the invention.

The diagrams depicted herein are illustrative. There can be many variations to the diagrams or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two- or three-digit reference numbers. With minor exceptions, the leftmost digit(s) of each reference number corresponds to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Embodiments of the present invention use a micro air and surface sprayer (“MASS”) to disperse a micro-aerosol, a gas-like fog, with particle size ranging from 0.1 microns to 2 microns in diameter. Micro-aerosols provide advantages over traditional foggers in that foggers typically have a particle size of from five to 50 microns, with an average size of 20 microns. Thus, a micro-aerosol is approximately twenty times smaller on average than a fog.

Since the micro-aerosol particles are small, they are widely dispersed using diffusion going from areas of high concentration to low concentration. The particles act like a gas and evenly distribute. It is unnecessary to “spray” or “fog” and labor is significantly reduced compared to traditional foggers and sprayers. As micro-aerosol particles average one micron, they clean both air and surface without getting surfaces wet or affecting electronics. When particles are less than 0.7 microns in diameter, they go through soft materials, such as clothing, drapes, and carpets. As they pass through fabrics and into carpeting, they are disinfecting where traditional fog particles have no access. The micro-aerosol particles kill odors, since they can get to bacteria, mold, and viruses present throughout the environment.

The MASS consistent with embodiments of the present invention disperses a micro-aerosol in ranges from about 0.1 to about 2.5 microns, with an average particle size of about 1 micron. Because of the particle size, the particles follow Graham's Law going from areas of high concentration to areas of low concentration diffusing through the environment and covering virtually all areas. By placing and adjusting a tortuous path within one or more misting nozzles through which generated particulates of size about 0.1 microns to about 200 microns must pass prior to ejection at an exterior portion of the MASS, the size of the particles within the micro-aerosol can be fine-tuned.

In exemplary embodiments consistent with the present invention, Paeroltye, a proprietary form of hypochlorous acid (“HOCL”) is used as the micro-aerosol. Paerolyte is an all-natural organic disinfectant that is eighty times stronger than bleach. Paerolyte is PH neutral, non-toxic to humans, and safe on skin, but it kills up to 99.9999% of mold, bacteria, and viruses with which it comes in contact. While Paerolyte is used in an exemplary embodiment of the invention, those skilled in the art having read this disclosure will appreciate that other forms of HOCL and other substances can be used with the MASS described herein. However, HOCL has benefits due to its use of phagocytosis that uses HOCL's neutral electrical charge to penetrate and destroy negatively charged cell walls of bacteria and viruses. The free-flowing particles of HOCL are attracted to the pathogens like a magnet, quickly attacking them where they then oxidize and are destroyed. Only particles in this size range can act like a gas and cover virtually all areas in an enclosed space. The MASS used in combination with HOCL produces a reduced pathogen environment within confined spaces. Thus, it is suitable for use in classrooms, hospitals, airplanes, offices, and any space needing cleansing.

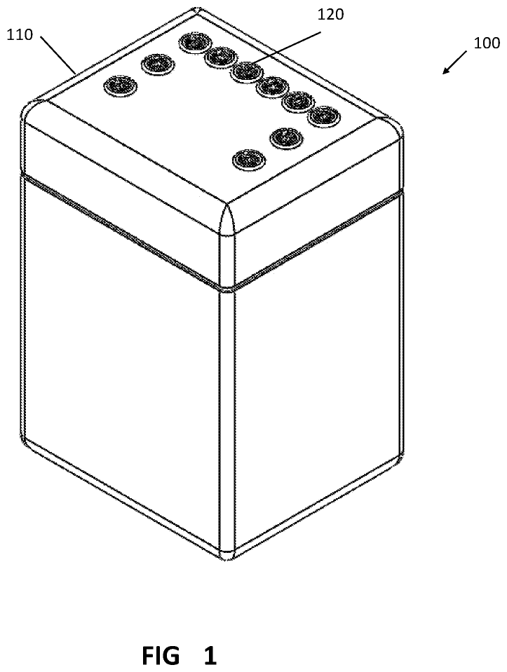

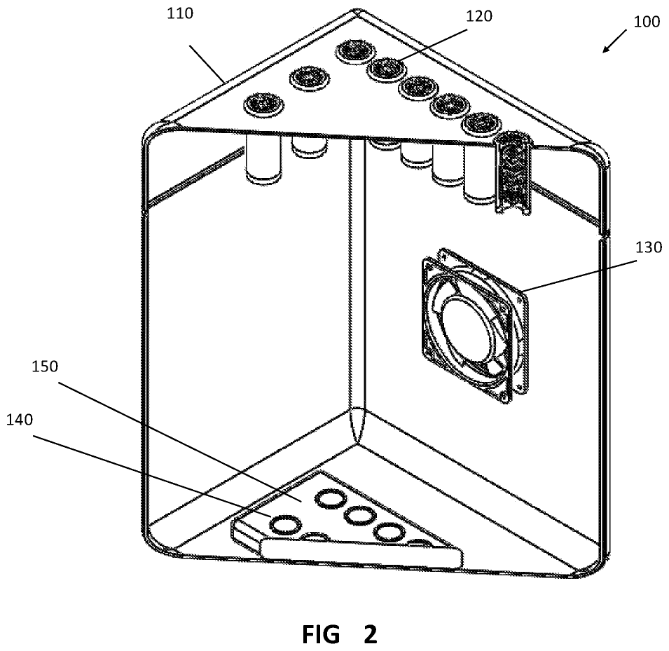

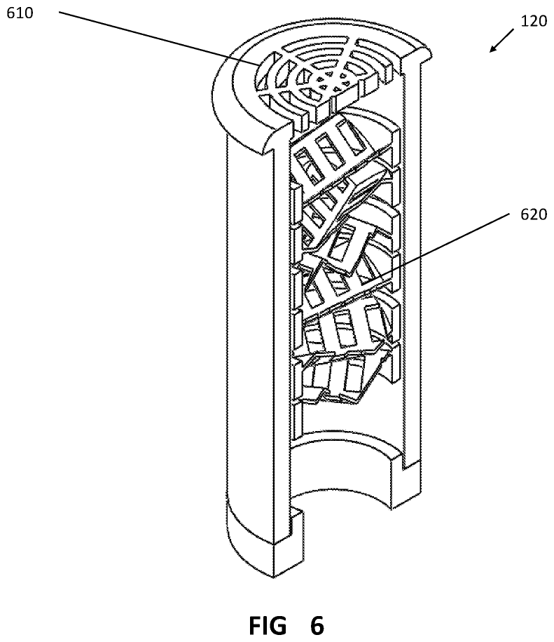

FIG. 1 depicts an orthogonal view of a MASS 100 according to an embodiment of the present invention. The MASS 100 comprises an enclosure 110 and one or more ejection nozzles 120 each comprising a tortuous path. The ejection nozzles 120 are described in detail in FIGS. 6-10. The enclosure holds HOCL fluid that is aerosolized within the enclosure 110 by an atomizer (shown in FIG. 2). A positive pressure device (shown in FIG. 2) generates a positive pressure within the enclosure 110 that forces the aerosolized HOCL through the ejection nozzles 120 into an exterior atmosphere.

FIG. 2 depicts a cutaway view of the MASS 100 according to an embodiment of the present invention. FIG. 2 illustrates the atomizer 150. In an exemplary embodiment of the invention, the atomizer 150 comprises one or more ultrasonic nozzles 140 embedded within the atomizer 150. In the exemplary embodiment the atomizer 150 floats about one inch below the surface of the HOCL within the enclosure 110. Thus, as the level of HOCL decreases through use, or increases as additional HOCL is added inside the enclosure 110, the atomizer remains in communication with the HOCL to generate an aerosolized particle mist.

The ultrasonic nozzles 140 aerosolize the HOCL into the aerosolized particle mist with particle size between about 0.1 to about 200 microns. Those skilled in the art after reading this disclosure will appreciate that other atomizers can be used in place of the one used in the exemplary embodiment. For example, an atomizer with air assist or an atomizer with hydraulic pressure may be used in place of the ultrasonic nozzles 140 used in the exemplary embodiment.

FIG. 2 further illustrates a positive pressure device 130 to generate a positive pressure that forces the aerosolized particle mist through the ejection nozzles 120 having a tortuous path. In an exemplary embodiment consistent with the present invention, the positive pressure device 130 is one or more fans. In an alternative embodiment, a compressor may be used as a positive pressure device 130. The positive pressure may be adjusted to adjust the size and number of particles that exit the ejection nozzles 120.

In an alternative embodiment, a negative pressure is created in the enclosure 110 by placing the ejection nozzles 120 before a fan 130 and having the fan 130 blowing out, drawing the particles out of the enclosure 110.

The atomizer 150 and the positive pressure device 130 may be powered by a battery pack (not shown) or by an external AC or DC power source (not shown). A timer (not shown) may be used to set a running time for the atomizer 150 and positive pressure device 130. In an alternative embodiment, relative humidity of the atmosphere in which the MASS is placed may be measured and a setpoint of, for example, between about 40% to about 90% may be set with the MASS set to run until that setpoint for relative humidity is reached.

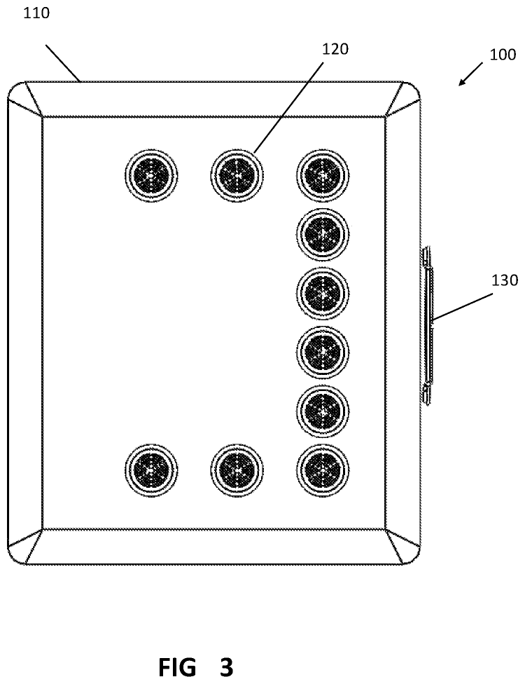

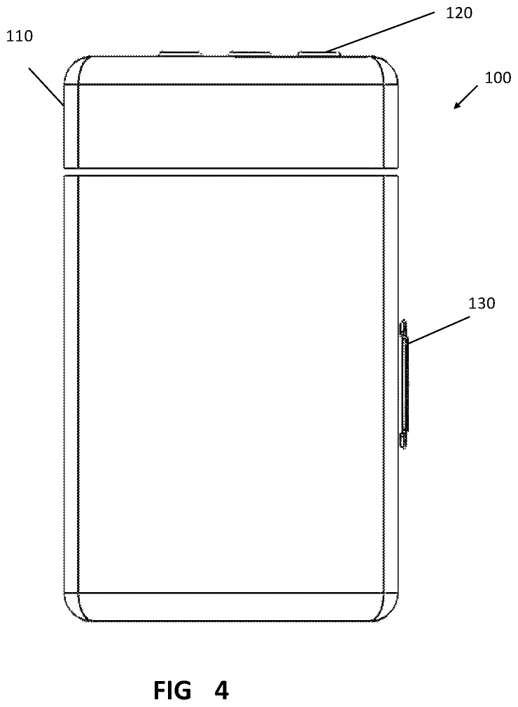

FIG. 3 is a top view of the MASS 100 according to an embodiment of the present invention and FIG. 4 is a side view of the MASS 100 according to an embodiment of the present invention. One can more easily see in FIG. 4 that the upper portion of enclosure 110 may be removable, for example, hinged, to add additional HOCL or service the interior components of the MASS. In areas with low relative humidity, such as desert or cooler climes, the MASS may be powered with the upper portion of enclosure 110 removed in order to more quickly bring up the relative humidity, prior to sealing the enclosure 110 and running the MASS in the conventional manner previously described.

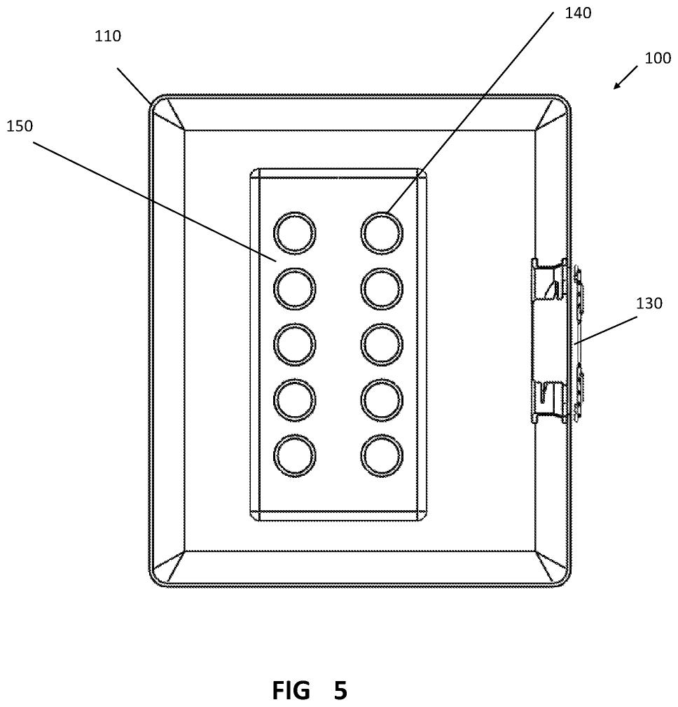

FIG. 5 is a top down, cut-away view of the MASS 100, showing the atomizer 150 with a plurality of ultrasonic nozzles 140. While ten ultrasonic nozzles 140 are illustrated, one of ordinary skill in the art after reading this disclosure will appreciate that any number and size may be chosen to achieve the desired particulate density within the enclosure 110.

FIG. 6 is an orthogonal, cut-away view of an ejection nozzle 120 having a tortuous path according to an embodiment of the present invention. The ejection nozzle comprises an ejection grate 610 and one or more internal grates 620. The internal grates comprise a plurality of slots and are placed to form a tortuous path that the aerosolized particle mist must pass through prior to ejection through the ejection grate 610. The tortuous path knocks out the particle sizes greater than about 2.5 microns, so that the micro-aerosol has a particle size of less than about 2.5 microns to about 0.1 microns. As previously mentioned, it is the positive pressure within the enclosure 110 that forces the aerosolized particle mist through the ejection nozzles 120 and into the atmosphere external to the enclosure 110. The number of internal grates 620 may be changed in order to produce a desired particle size for the micro-aerosol. The greater the number of internal grates 620, for example, the smaller the particles in the micro-aerosol. While the exemplary embodiment uses one or more internal grates 620 to form the tortuous path, those of ordinary skill in the art will appreciate after reading this disclosure that other mechanisms and designs can be used to form the tortuous path.

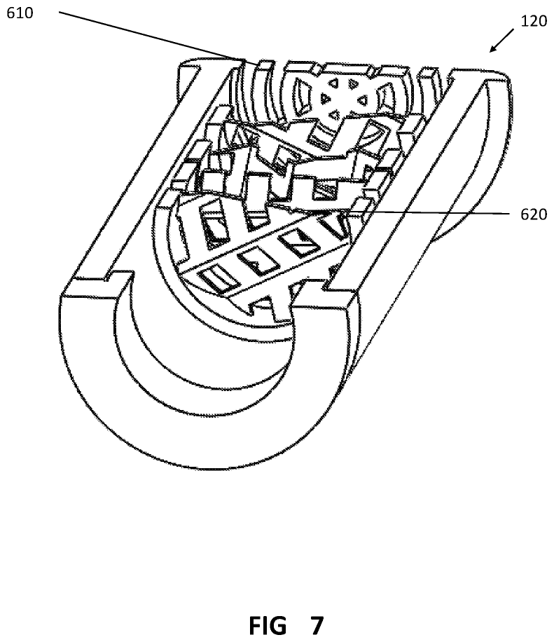

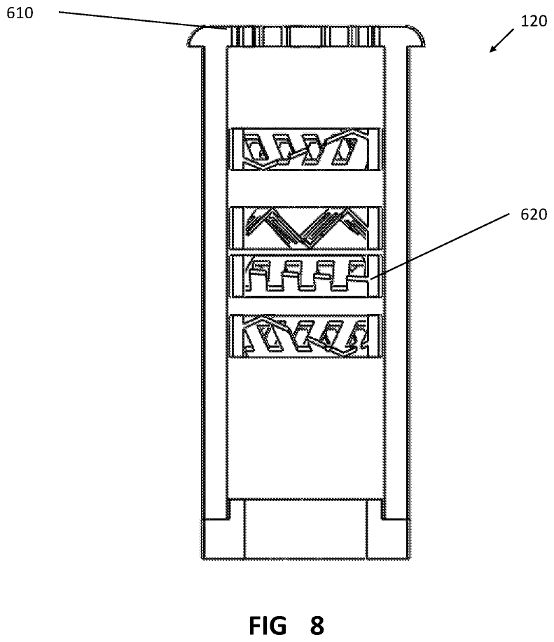

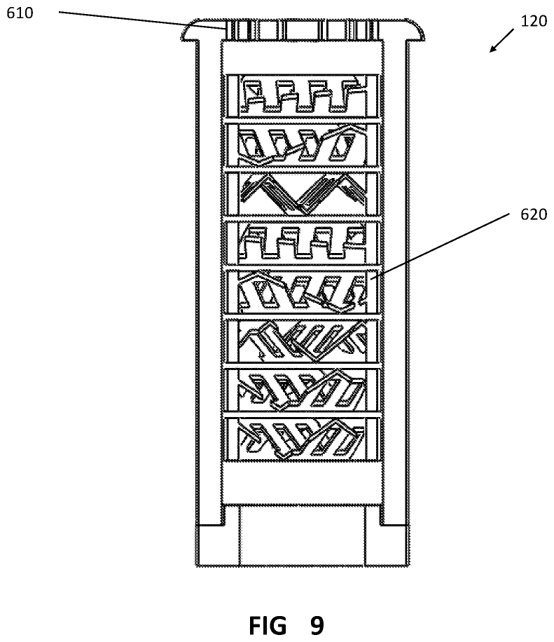

FIG. 7 is an additional orthogonal, cut-away view of an ejection nozzle 120 having four internal grates 620 according to an embodiment of the present invention. FIGS. 8 and 9 are cut-away view of ejection nozzles 120 having four and eight internal grates 620, respectively, according to embodiments of the present invention.

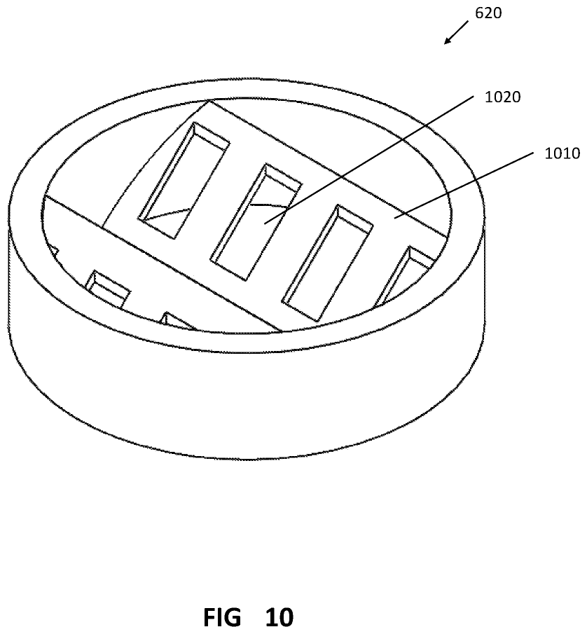

FIG. 10 is an orthogonal view of the internal grate 620 according to an embodiment of the present invention. The internal grate 620 comprise one or more grate plates 1010, with each grate plate 1010 having one or more slots 1020. Embodiments of the internal grate 620 may also include a grate plate 1010 having zero slots 1020. Those skilled in the art after reading this specification will appreciate the variability that could be implemented in both the number of grate plates 1010 and the number of slots 1020 in an internal grate 620.

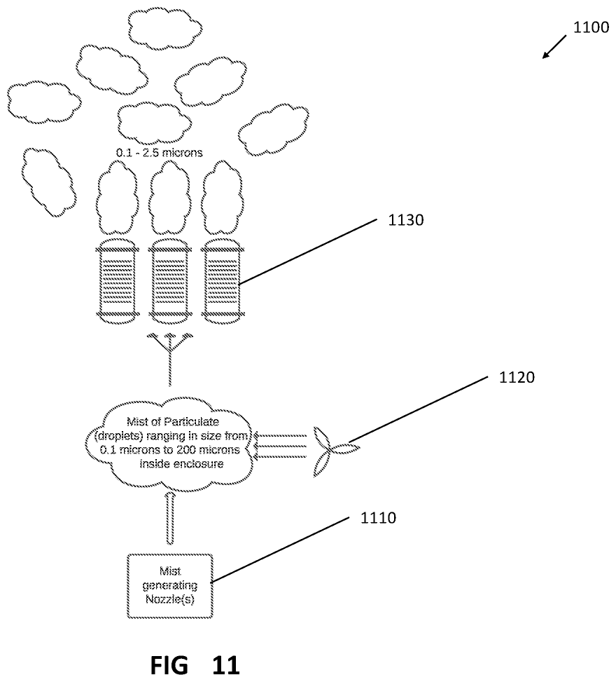

FIG. 11 is a piping and instrumentation diagram 1100 of a MASS 100 according to an embodiment of the present invention. The atomizer, shown as mist generating nozzle 1110, generates an aerosolized particle mist within the enclosure. A pressure source 1120 supplies pressure within the enclosure that forces the aerosolized particle mist through the ejection nozzles 1130 to generate a micro-aerosol with particle size from about 0.1 micron to about 2.5 microns.

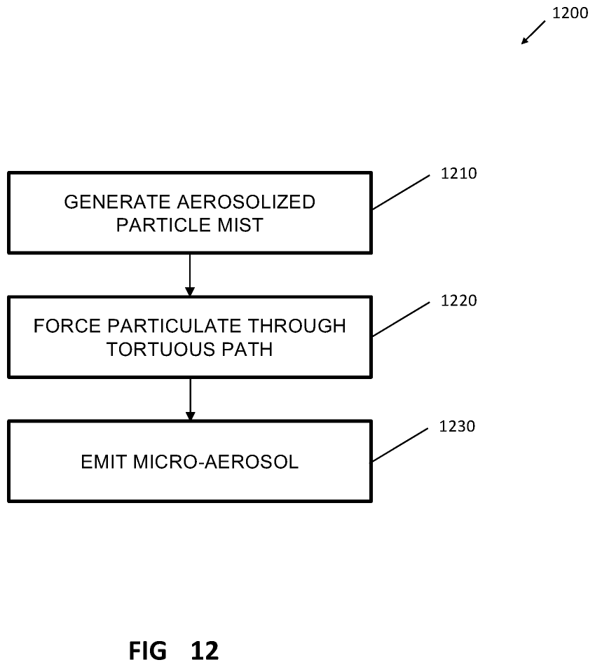

FIG. 12 is a flowchart of a method 1200 for generating a micro-aerosol according to an embodiment of the invention. An aerosolized particle mist is generated (stage 1210). The aerosolized particle mist is forced through a tortuous path (stage 1220) to eliminate particle sizes larger than about 2.5 microns. In an exemplary embodiment, the forcing happens by having a positive pressure within an enclosure in which the aerosolized particle mist is generated. Varying the pressure will vary the size and number of particles that exit the enclosure. The resulting micro-aerosol is emitted into the atmosphere (stage 1230).

The flowchart and block diagrams in the Figures illustrate the architecture, functionality, and operation of possible implementations of systems and methods according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A system, comprising:
an enclosure for containing a solution;
an atomizer in communication with an interior of the enclosure and the solution for generating an aerosolized particle mist within the interior of the enclosure;
a positive pressure device in communication with the interior of the enclosure for generating a positive pressure within the enclosure relative to an atmosphere outside of the enclosure; and
an ejection nozzle having a tortuous path, wherein an entrance of the tortuous path is in communication with the interior of the enclosure and an exit of the tortuous path is in communication with the atmosphere outside of the enclosure.

2. The system of claim 1, wherein the atomizer generates particles from the solution having a particle size between about 0.1 microns and about 200 microns.

3. The system of claim 1, wherein the atomizer floats in an upper region of the solution within the enclosure.

4. The system of claim 1, wherein the atomizer comprises an ultrasonic nozzle.

5. The system of claim 1, wherein the ejection nozzle comprises an internal grate that forms at least a portion of the tortuous path.

6. The system of claim 1, wherein the ejection nozzle comprises a plurality of internal grates that form at least a portion of the tortuous path.

7. The system of claim 1, wherein the ejection nozzle ejects particles from the aerosolized particle mist with sizes between about 0.1 micron and about 2.5 microns.

8. The system of claim 1, wherein the positive pressure device provides a variable positive pressure within the enclosure.

9. The system of claim 1, further comprising a timer that turns off the atomizer after a predetermined period of time.

10. The system of claim 1, further comprising a relative humidity sensor for sensing relative humidity in the atmosphere outside of the enclosure.

11. The system of claim 10, further comprising a controller for setting and controlling the relative humidity in the atmosphere outside of the enclosure.

12. A method of providing a micro-aerosol to an atmosphere, comprising:
generating an aerosolized particle mist within an enclosure;
providing a positive pressure to the aerosolized particle mist within the enclosure; and
eliminating a substantial majority of particles larger than about 2.5 microns from the aerosolized particle mist using a tortuous path prior to releasing the aerosolized particle mist into the atmosphere as the micro-aerosol.

13. The method of claim 12, further comprising:
setting a desired relative humidity for the atmosphere; and
releasing the micro-aerosol into the atmosphere until the desired relative humidity is reached.

14. A system, comprising:
an enclosure for containing a solution;
an atomizer in communication with an interior of the enclosure and the solution for generating an aerosolized particle mist within the interior of the enclosure;
an ejection nozzle having a tortuous path, wherein an entrance of the tortuous path is in communication with the interior of the enclosure and an exit of the tortuous path is in communication with the atmosphere outside of the enclosure; and
a negative pressure device in communication with the exit of the tortuous path for generating a negative pressure to draw the aerosolized particle mist out of the tortuous path and into the atmosphere outside of the enclosure.

15. The system of claim 14, wherein the atomizer generates particles from the solution having a particle size between about 0.1 microns and about 200 microns.

16. The system of claim 14, wherein the atomizer comprises an ultrasonic nozzle.

17. The system of claim 14, wherein the ejection nozzle comprises an internal grate that forms at least a portion of the tortuous path.

18. The system of claim 14, wherein the ejection nozzle comprises a plurality of internal grates that form at least a portion of the tortuous path.

19. The system of claim 14, wherein the ejection nozzle ejects particles from the aerosolized particle mist with sizes between about 0.1 micron and about 2.5 microns.

20. The system of claim 14, wherein the positive pressure device provides a variable positive pressure within the enclosure.
